(19) United States
(12) Patent Application Publication
(10) Pub. No.: US 2006/0148876 A1
(43) Pub. Date: Jul. 6, 2006

(54) Nr3b1 nuclear receptor binding 3-substituted pyrazoles

(76) Inventors: Ulrich Deuschle, Bammental (DE); Stefanie Heck, Heidelberg (DE); Ingo Kober, Gaiberg (DE); Ulrike Bauer, Sandhausen (DE); Imola Balogh, Weinheim (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/526,940

(22) PCT Filed: Jul. 2, 2003
(86) PCT No.: PCT/EP03/07066

§ 371 Date: Oct. 21, 2005

(30) Foreign Application Priority Data
Sep. 10, 2002 (EP) 02020256.0

(51) Int. Cl. A61K 31/416; A61K 31/415; C07D 405/02; C07D 231/12

(52) U.S. Cl. 514/406; 548/377.1; 548/365.7

(57) ABSTRACT

The present invention relates to compounds according to the general formula (I) which bind to the NR3B1 receptor and act as antagonists or agonists of the NR3B1 receptor. The invention further relates to the treatment of diseases and/or conditions through binding of said nuclear receptor by said compounds and the production of medicaments using said compounds.

The present invention relates to compounds according to the general formula (1) which bind to the NR3B1 receptor and act as antagonists or agonists of the NR3B1 receptor. The invention further relates to the treatment of diseases and/or conditions through binding of said nuclear receptor by said compounds and the production of medicaments using said compounds.

### BACKGROUND OF THE INVENTION

Multicellular organisms are dependent on advanced mechanisms of information transfer between cells and body compartments. The information that is transmitted can be highly complex and can result in the alteration of genetic programs involved in cellular differentiation, proliferation, or reproduction. The signals, or hormones, are often simple molecules, such as peptides, fatty acid, or cholesterol derivatives.

Many of these signals produce their effects by ultimately changing the transcription of specific genes. One well-studied group of proteins that mediate a cell's response to a variety of signals is the family of transcription factors known as nuclear receptors, hereinafter referred to often as “NR”. Members of this group include receptors for steroid hormones, vitamin D, ecdysone, cis and trans retinoic acid, thyroid hormone, bile acids, cholesterol-derivatives, fatty acids (and other peroxisomal proliferators), as well as so-called orphan receptors, proteins that are structurally similar to other members of this group, but for which no ligands are known (Escriva, H. et al., Ligand binding was acquired during evolution of nuclear receptors, PNAS, 94, 6803-6808, 1997). Orphan receptors may be indicative of unknown signalling pathways in the cell or may be nuclear receptors that function without ligand activation. The activation of transcription by some of these orphan receptors may occur in the absence of an exogenous ligand and/or through signal transduction pathways originating from the cell surface (Mangelsdorf, D. J. et al., The nuclear receptor superfamily: the second decade, Cell 83, 835-839, 1995).

In general, three functional domains have been defined in NRs. An amino terminal domain is believed to have some regulatory function. A DNA-binding domain hereinafter referred to as “DBD” usually comprises two zinc finger elements and recognises a specific Hormone Responsive Element hereinafter referred to as “HRE” within the promoters of responsive genes. Specific amino acid residues in the “DBD” have been shown to confer DNA sequence binding specificity (Schena, M. & Yamamoto, K. R., Mammalian Glucocorticoid Receptor Derivatives Enhance Transcription in Yeast, Science, 241:965-967, 1988). A Ligand-binding-domain hereinafter referred to as “LBD” is at the carboxy-terminal region of known NRs. In the absence of hormone, the LBD appears to interfere with the interaction of the DBD with its BRE. Hormone binding seems to result in a conformational change in the NR and thus opens this interference (Brzozowski et al., Molecular basis of agonism and antagonism in the oestogen receptor, Nature, 389, 753-758, 1997; Wagner et al., A structural role for hormone in the thyroid hormone receptor, Nature, 378, 690-697. 1995). A NR without the HBD constitutively activates transcription but at a low level.

Coactivators or transcriptional activators are proposed to bridge between sequence specific transcription factors, the basal transcription machinery and in addition to influence the chromatin structure of a target cell. Several proteins like SRC-1, ACTR, and Grip1 interact with NRs in a ligand enhanced manner (Heery et al., A signature motif in transcriptional coactivators mediates binding to nuclear receptors, Nature, 387, 733-736; Heinzel et al., A complex containing N—CoR, mSin3 and histone deacetylase mediates transcriptional repression, Nature 387, 43-47, 1997). Furthermore, the physical interaction with negative receptor-interacting proteins or corepressors has been demonstrated (Xu et al., Coactivator and Corepressor complexes in nuclear receptor function, Curr Opin Genet Dev, 9 (2), 140-147, 1999).

Nuclear receptor modulators like steroid hormones affect the growth and function of specific cells by binding to intracellular receptors and forming nuclear receptor-ligand complexes. Nuclear receptor-hormone complexes then interact with a hormone response element (HRE) in the control region of specific genes and alter specific gene expression.

The Estrogen Related Receptor (ERR) alpha, beta and gamma (hereinafter referred to as NR3B1, NR3B2 and NR3B3 when referring to the human receptor) are nuclear receptors which activate genes upon binding to the promoter region of target genes either in a homodimeric or monomeric fashion or as a heterodimer with the estrogen receptor alpha. NR3B1 and NR3B2 were the first orphan nuclear receptors identified more than a decade ago (Giguere, et al. 1988 Nature 331, 91-94). Although the ERR's display high homology to the estrogen receptor (ER) they do not bind or respond to natural occurring estrogens. The ERR's are structurally and functionally related to the ERa and ERb and have been shown to posses the potential to positively and negatively regulate estrogen regulated gene networks (Vanacker, J M et al 1999 EMBO J. 18, 4270-4279).

The ERR's are described as constitutive activators of transcription. They contain a well conserved AF-2 domain that is necessary for the constitutive transcriptional activity. The interaction of the ERR's with cofactors (e.g. GRIP-1, SRC-1, ACTR) is ligand independent. Nevertheless, the observation that the constitutive activity depends on a factor present in serum that can be withdrawn gives a hint, that there may exist also agonistic ligands for the ERR's that can induce their activity.

To date no physiological ligands have been identified for ERRa (NR3B1) although in very recent publications, Diethylstilbestrol was identified as a synthetic substance able to bind to and inhibit the constitutive activity of ERRa but also ERRb and ERRg (Tremblay, G. B. 2001 Genes & Development 15, 833-838).

To date only very few compounds have been described which bind the NR3B1 receptor and thus show utility for treating diseases or conditions which are due to or are influenced by said nuclear receptor (Maloney at al., J Med Chem, 10; 43(16):2971-4, 2000).

It was thus an object of the present invention to provide for novel NR3B1 binding compounds. It was thus an object of the present invention to provide for compounds which by means of binding the NR3B1 receptor act as antagonist or agonist of said receptor and thus show utility for treating diseases or conditions which are due to or influenced by said nuclear receptor.

It was further an object of the invention to provide for compounds which may be used for the manufacture of a medicament for the treatment of conditions or diseases like cancer, osteoporosis, obesity, lipid disorders, cardiovascular disorders or fertility and reproductive health associated conditions or diseases. In a preferred embodiment of the invention it was an object of the invention to provide for compounds for the manufacture of anti-tumour medicaments.

### SUMMARY OF THE INVENTION

The present invention provides, inter alia, novel NR3B1 nuclear receptor protein binding compounds according to the general formula (1) shown below. Said compounds are also binders of mammalian homologues of said receptor. Further the object of the invention was solved by providing for amongst the NR3B1 nuclear receptor protein binding compounds according to the general formula (1) such compounds which act as antagonists and such compounds which act as agonists of the human ERRa receptor or a mammalian homologue thereof.

The invention provides for ERRa antagonists or agonists which may be used for the manufacture of a medicament for the treatment of cancer, osteoporosis, obesity, lipid disorders or a cardiovascular disorder or fertility and reproductive health associated conditions or diseases. In a preferred embodiment of the invention the compounds according to the invention may be used for manufacture of anti-tumour medicaments and/or for the treatment of diseases such as cancer.

The foregoing merely summarises certain aspects of the present invention and is not intended, nor should it be construed, to limit the invention in any manner. All patents and other publications recited herein are hereby incorporated by reference in their entirety.

### DETAILED DESCRIPTION OF THE PREFERRED EMBODIMENTS

The invention provides for a compound according to formula (1), or pharmaceutical acceptable salts or solvates thereof, hereinafter also referred to as the “compounds according to the invention” including particular and preferred embodiments thereof, 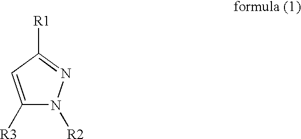
wherein in formula (1) as shown above,
R₁ is phenyl, substituted phenyl, C₅ to C₆ heteroaryl, C₅ to C₆ substituted heteroaryl, napthyl or substituted napthyl,
R₂ is H, C₁ to C₈ akyl, C₁ to C₇ acyl or C₁ to C₇ substituted acyl, C₁ to C₈ substituted alkyl, C₇ to C₁₂ alkylphenyl or C₇ to C₁₂ substituted phenylalkyl, C₃ to C₈ cycloalkyl, C₃ to C₈ substituted cycloalkyl, C₅ to C₆ heteroaryl, [C₅ to C₆]-heteroaryl-(C₁ to C₆)-alkyl, and
R₃ is H, C₁ to C₈ alkyl, C₁ to C₈ substituted alkyl, C₇ to C₁₂ alkylphenyl or or C₇ to C₁₂ substituted phenylalkyl, halogen, C₁ to C₈ alkoxy, furanyl, substituted furanyl, thiazyl, substituted thiazyl, carboxy, ester, amide or C₁ to C₈ aminoacyl.

The inventors have unexpectedly identified the compounds as well as the general structure capable of effectively binding ERRa and as claimed in the present invention amongst approximately 4500 compounds that were within a compound library that was not previously disclosed

The compounds of the invention can also exist as solvates and hydrates. Thus, these compounds may crystallise with, for example, waters of hydration, or one, a number of, or any fraction thereof of molecules of the mother liquor solvent. The solvates and hydrates of such compounds are included within the scope of this invention.

The term “halogen” refers to the fluoro, chloro, bromo or iodo atoms. There can be one or more halogen, which are the same or different. Preferred halogens are chloro and fluoro.

The symbol “H” denotes a hydrogen atom.

The term “C₁ to C₇ acyl” encompasses groups such as formyl, acetyl, propionyl, butyryl, pentanoyl, pivaloyl, hexanoyl, heptanoyl, benzoyl and the like. Preferred acyl groups are acetyl and benzoyl.

The term “C₁ to C₇ substituted acyl” denotes the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, C₁ to C₆ alkyl ester, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted acyl groups may be substituted once or more, and preferably once or twice, with the same or with different substituents.

Examples of C₁ to C₇ substituted acyl groups include 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl and the like.

The term “substituted phenyl” specifies a phenyl group substituted with one or more, and preferably one or two, moieties chosen from the groups consisting of halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted, such that, for example, a biphenyl results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3 or 4-hydroxyphenyl, 2,4-dihydroxyphenyl, the protected-hydroxy derivatives thereof and the like; a nitrophenyl group such as 2, 3 or 4-nitrophenyl; a cyanophenyl group, for example, 2, 3 or 4-cyanophenyl; a mono- or di(alkyl)phenyl group such as 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl and the like; a mono or di(alkoxyl)phenyl group, for example, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3, or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like.

The term “heteroaryl” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms. Examples of heteroaryls include pyridinyl, pyrimidinyl, and pyrazinyl, pyridazinyl, pyrrolo, furano, thiopheno, oxazolo, isoxazolo, phthalimido, thiazolo and the like.

The term “substituted heteroaryl” means the above-described heteroaryl is substituted with, for example, one or more, and preferably one or two, substituents which are the same or different which substituents can be halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino groups.

The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino.

Examples of the term “substituted naphthyl” includes a mono or di(halo)naphthyl group such as 1, 2, 3, 4, 5, 6, 7 or 8-chloronaphthyl, 2,6-dichloronaphthyl, 2,5-dichloronaphthyl, 3,4-dichloronaphthyl, 1, 2, 3, 4, 5, 6, 7 or 8-bromonaphthyl, 3,4-dibromonaphthyl, 3-chloro-4-fluoronaphthyl, 1, 2, 3, 4, 5, 6, 7 or 8-fluoronaphthyl and the like; a mono or di(hydroxy)naphthyl group such as 1, 2, 3, 4, 5, 6, 7 or 8-hydroxynaphthyl, 2,4-dihydroxynaphthyl, the protected-hydroxy derivatives thereof and the like; a nitronaphthyl group such as 3- or 4-nitronaphthyl; a cyanonaphthyl group, for example, 1, 2, 3, 4, 5, 6, 7 or 8-cyanonaphthyl; a mono- or di(alkyl)naphthyl group such as 2, 3, 4, 5, 6, 7 or 8-methylnaphthyl, 1,2,4-dimethylnaphthyl, 1, 2, 3, 4, 5, 6, 7 or 8-(isopropyl)naphthyl, 1, 2, 3, 4, 5, 6, 7 or 8-ethylnaphthyl, 1, 2, 3, 4, 5, 6, 7 or 8-(n-propyl)naphthyl and the like; a mono or di(alkoxy)naphthyl group, for example, 2,6-dimethoxynaphthyl, 1, 2, 3, 4, 5, 6, 7 or 8-methoxynaphthyl, 1, 2, 3, 4, 5, 6, 7 or 8-ethoxynaphthyl, 1, 2, 3, 4, 5, 6, 7 or 8-(isopropoxy)naphthyl, 1, 2, 3, 4, 5, 6, 7 or 8-(t-butoxy)naphthyl, 3-ethoxy-4-methoxynaphthyl and the like; 1, 2, 3, 4, 5, 6, 7 or 8-trifluoromethylnaphthyl; a mono- or dicarboxynaphthyl or (protected carboxy)naphthyl group such as 1, 2, 3, 4, 5, 6, 7 or 8-carboxynaphthyl or 2,4-di(-protected carboxy)naphthyl; a mono- or di(hydroxymethyl)naphthyl or (protected hydroxymethyl)naphthyl such as 1, 2, 3, 4, 5, 6, 7 or 8-(protected hydroxymethyl)naphthyl or 3,4-di(hydroxymethyl)naphthyl; a mono- or di(amino)naphthyl or (protected amino)naphthyl such as 1, 2, 3, 4, 5, 6, 7 or 8-(amino)naphthyl or 2,4-(protected amino)-naphthyl, a mono- or di(aminomethyl)naphthyl or (protected aminomethyl)naphthyl such as 2, 3, or 4-(aminomethyl)naphthyl or 2,4-(protected aminomethyl)-naphthyl; or a mono- or di-(N-methylsulfonylamino) naphthyl such as 1, 2, 3, 4, 5, 6, 7 or 8-(N-methylsulfonylamino)naphthyl. Also, the term “substituted naphthyl” represents disubstituted naphthyl groups wherein the substituents are different, for example, 3-methyl-4-hydroxynaphth-1-yl, 3-chloro-4-hydroxynaphth-2-yl, 2-methoxy-4-bromonaphth-1-yl, 4-ethyl-2-hydroxynaphth-1-yl, 3-hydroxy-4-nitronaphth-2-yl, 2-hydroxy-4-chloronaphth-1-yl, 2-methoxy-7-bromonaphth-1-yl, 4-ethyl-5-hydroxynaphth-2-yl, 3-hydroxy-8-nitronaphth-2-yl, 2-hydroxy-5-chloronaphth-1-yl and the like.

The term “C₁ to C₈ alkyl” denotes such radicals as methyl, ethyl, n-propyl, isopropyl, n-butyl, iso-butyl, sec-butyl, tert-butyl, amyl, tert-amyl, hexyl, n-heptyl, 2-heptyl, 3-heptyl, 4-heptyl, 2-methyl-1hexyl, 2-methyl-2hexyl, 2-methyl-3-hexyl, n-octyl and the like.

The term “C₁ to C₈ substituted alkyl” denotes that the above C₁ to C₈ alkyl groups are substituted by one or more, and preferably one or two, halogen, hydroxy, protected hydroxy, oxo, protected oxo, C₃ to C₇ cycloalkyl, naphthyl, amino, protected amino, (monosubstituted)amino, protected (monosubstituted)amino, (disubstituted)amino, guanidino, protected guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted alkyl groups may be substituted once or more, and preferably once or twice, with the same or with different substituents.

Examples of the above substituted alkyl groups include the 2-oxo-prop-1-yl, 3-oxo-but-1-yl, cyanomethyl, nitromethyl, chloromethyl, hydroxymethyl, tetrahydropyranyloxymethyl, trityloxymethyl, propionyloxymethyl, amino, methylamino, aminomethyl, dimethylamino, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl, N-acetyl-1-aminoethyl and the like.

The term “C₇ to C₁₂ phenylalkyl” denotes a C₁ to C₆ alkyl group substituted at any position by a phenyl, substituted phenyl, heteroaryl or substituted heteroaryl. Examples of such a group include benzyl, 2-phenylethyl, 3-phenyl(n-propyl), 4-phenylhexyl, 3-phenyl(n-amyl), 3-phenyl(sec-butyl) and the like. Preferred C₇ to C₁₂ phenylalkyl groups are the benzyl and the phenylethyl groups.

The term “C₇ to C₁₂ substituted phenylalkyl” denotes a C₇ to C₁₂ phenylalkyl group substituted on the C₁ to C₆ alkyl portion with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, protected guanidino, heterocyclic ring, substituted heterocyclic ring, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N,N—(C₁ to C₆ dialkyl)carboxamide, cyano, N—(C₁ to C₆ alkylsulfonyl)amino, thiol, C₁ to C₄ alkylthio, C₁ to C₄ alkylsulfonyl groups; and/or the phenyl group may be substituted with one or more, and preferably one or two, substituents chosen from halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl) carboxamide, protected N—(C₁ to C₆ alkyl) carboxamide, N,N-di(C₁ to C₆ alkyl)carboxamide, trifluoromethyl, N—((C₁ to C₆ alkyl)sulfonyl)amino, N-(phenylsulfonyl)amino, cyclic C₂ to C₇ alkylene or a phenyl group, substituted or unsubstituted, for a resulting biphenyl group. The substituted alkyl or phenyl groups may be substituted with one or more, and preferably one or two, substituents which can be the same or different.

Examples of the term “C₇ to C₁₂ substituted phenylalkyl” include groups such as 2-phenyl-1-chloroethyl, 2-(4-methoxyphenyl)ethyl, 4-(2,6-dihydroxy phenyl)n-hexyl, 2-(5-cyano-3-methoxyphenyl)n-pentyl, 3-(2,6-dimethylphenyl)n-propyl, 4-chloro-3-aminobenzyl, 6-(4-methoxyphenyl)-3-carboxy(n-hexyl), 5-(4-aminomethylphenyl)-3-(aminomethyl)n-pentyl, 5-phenyl-3-oxo-n-pent-1-yl and the like.

The term “heterocycle” or “heterocyclic ring” denotes optionally substituted three-membered to eight-membered rings that have 1 to 4 heteroatoms, such as oxygen, sulfur and/or nitrogen, in particular nitrogen, either alone or in conjunction with sulfur or oxygen ring atoms. These three-membered to eight-membered rings may be saturated, fully unsaturated or partially unsaturated, with fully saturated rings being preferred. Preferred heterocyclic rings include morpholino, piperidinyl, piperazinyl, 2-amino-imidazoyl, tetrahydrofurano, pyrrolo, tetrahydrothiophen-yl, hexylmethyleneimino and heptylmethyleneimino.

The term “substituted heterocycle” or “substituted heterocyclic ring” means the above-described heterocyclic ring is substituted with, for example, one or more, and preferably one or two, substituents which are the same or different which substituents can be halogen, hydroxy, protected hydroxy, oxo, protected oxo, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino, heterocycle or substituted heterocycle groups.

The term “C₁ to C₈ alkoxy” as used herein denotes groups such as methoxy, ethoxy, n-propoxy, isopropoxy, n-butoxy, t-butoxy and like groups. A preferred alkoxy is methoxy. The term “C₁ to C₈ substituted alkoxy” means the alkyl portion of the alkoxy can be substituted in the same manner as in relation to C₁ to C₈ substituted alkyl. [Please check carefully . . . ]

The term “C₁ to C₈ aminoacyl” encompasses groups such as formyl, acetyl, propionyl, butyryl, pentanoyl, pivaloyl, hexanoyl, heptanoyl, octanoyl, benzoyl and the like attached to a nitrogen moiety.

The term “C₁ to C₈ substituted aminoacyl” denotes the acyl group, attached to a nitrogen moiety, substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, C₁ to C₁₂ alkyl ester, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted acyl groups may be substituted once or more, and preferably once or twice, with the same or with different substituents.

Examples of C₁ to C₈ substituted acyl groups include 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

This invention provides a pharmaceutical composition comprising an effective amount of a compound according to the invention. Such compounds can be administered by various routes, for example oral, subcutaneous, intramuscular, intravenous or intracerebral. The preferred route of administration would be oral at daily doses of the compound for adult human treatment of about 0.01-5000 mg, preferably 1-1500 mg per day. The appropriate dose may be administered in a single dose or as divided doses presented at appropriate intervals for example as two, three four or more subdoses per day.

For preparing pharmaceutical compositions containing compounds of the invention, inert, pharmaceutically acceptable carriers are used. The pharmaceutical carrier can be either solid or liquid. Solid form preparations include, for example, powders, tablets, dispersible granules, capsules, cachets, and suppositories.

A solid carrier can be one or more substances which can also act as diluents, flavoring agents, solubilizers, lubricants, suspending agents, binders, or tablet disintegrating agents; it can also be an encapsulating material.

In powders, the carrier is generally a finely divided solid which is in a mixture with the finely divided active component. In tablets, the active compound is mixed with the carrier having the necessary binding properties in suitable proportions and compacted in the shape and size desired.

For preparing pharmaceutical composition in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter and the like.

The pharmaceutical compositions can include the formulation of the active compound with encapsulating material as a carrier providing a capsule in which the active component (with or without other carriers) is surrounded by a carrier, which is thus in association with it. In a similar manner, cachets are also included. Tablets, powders, cachets, and capsules can be used as solid dosage forms suitable for oral administration.

Liquid pharmaceutical compositions include, for example, solutions suitable for oral or parenteral administration, or suspensions, and emulsions suitable for oral administration. Sterile water solutions of the active component or sterile solutions of the active component in solvents comprising water, ethanol, or propylene glycol are examples of liquid compositions suitable for parenteral administration.

Sterile solutions can be prepared by dissolving the active component in the desired solvent system, and then passing the resulting solution through a membrane filter to sterilise it or, alternatively, by dissolving the sterile compound in a previously sterilised solvent under sterile conditions.

In one embodiment of the present invention a compound is provided according to formula (2) below, or pharmaceutically acceptable salts or solvates thereof, wherein R₂ is H, C₁ to C₈ alkyl, C₁ to C₈ substituted alkyl, C₇ to C₁₂ alkylphenyl or C₇ to C₁₂ substituted phenylalkyl, R₄ is H, C₁ to C₈ alkyl, halogen, C₁ to C₈ alkoxy, carboxy, ester, amide or C₁ to C₈ aminoacyl, and R₅ is H, C₁ to C₈ alkyl, halogen, C₁ to C₈ alkoxy, carboxy, ester, amide or C₁ to C₈ aminoacyl.

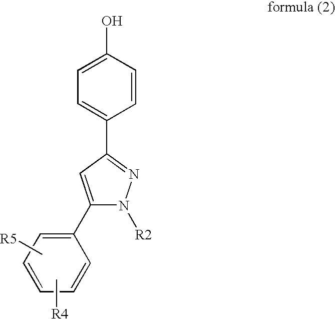

In a preferred embodiment of the invention a compound which may act as an antagonist of NR3B1 according to formula (3) is provided, or pharmaceutical acceptable salts or solvates thereof, wherein R₂ is H, C₁ to C₇ acyl or C₁ to C₇ substituted acyl, phenyl, substituted phenyl, C₅ to C₆ heteroaryl, C₅ to C₆ substituted heteroaryl, napthyl or substituted napthyl, and R₆ is H, C₁ to C₈ alkyl, C₁ to C₈ substituted alkyl, C₇ to C₁₂ alkylphenyl or C₇ to C₁₂ substituted phenylalkyl, carboxy, ester, amide or C₁ to C₈ aminoacyl.

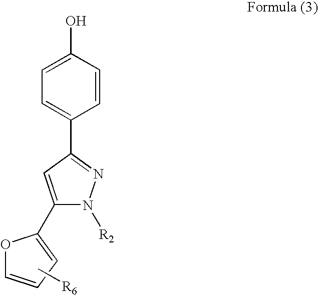

An other preferred compound which may act as antagonist of NR3B1 is shown in formula (4) below. The inventors have been able to demonstrate that the compound according to formula (4), wherein R₂ is H, C₁ to C₇ acyl or C₁ to C₇ substituted acyl, phenyl, substituted phenyl, C₅ to C₆ heteroaryl, C₅ to C₆ substituted heteroaryl, napthyl or substituted napthyl, and R₆ is H, C₁ to C₈ alkyl, C₁ to C₈ substituted alkyl, C₇ to C₁₂ alkylphenyl or C₇ to C₁₂ substituted phenylalkyl, carboxy, ester, amide or C₁ to C₈ aminoacyl has a low effective concentration at NR3B1 with an EC₅₀ of xxx μM wherein the EC₅₀ reflects the half-maximal effective concentration, and which is higher than the EC₅₀ of 1 μM for the published ERR antagonist DES (Tremblay G B et al., Genes & Development 15, 833-838, 2001).

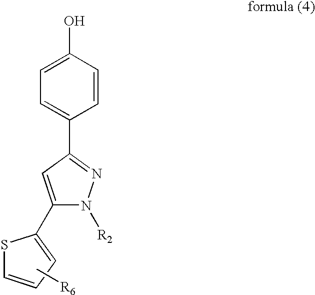

The inventors have also found the compounds according to formula (5 and 6) (shown below) to be active as antagonist of the NR3B1 human nuclear receptor (see figures for details).

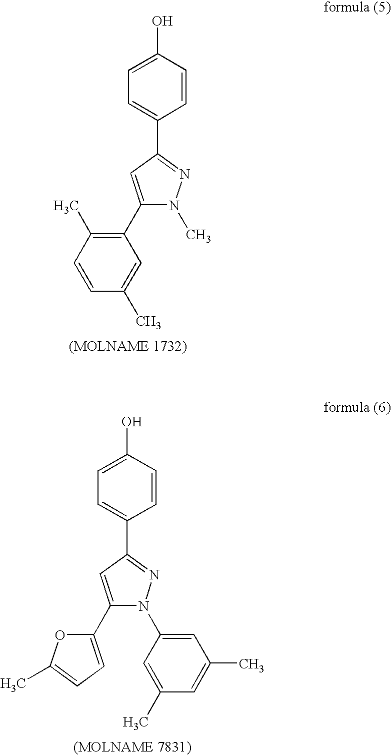

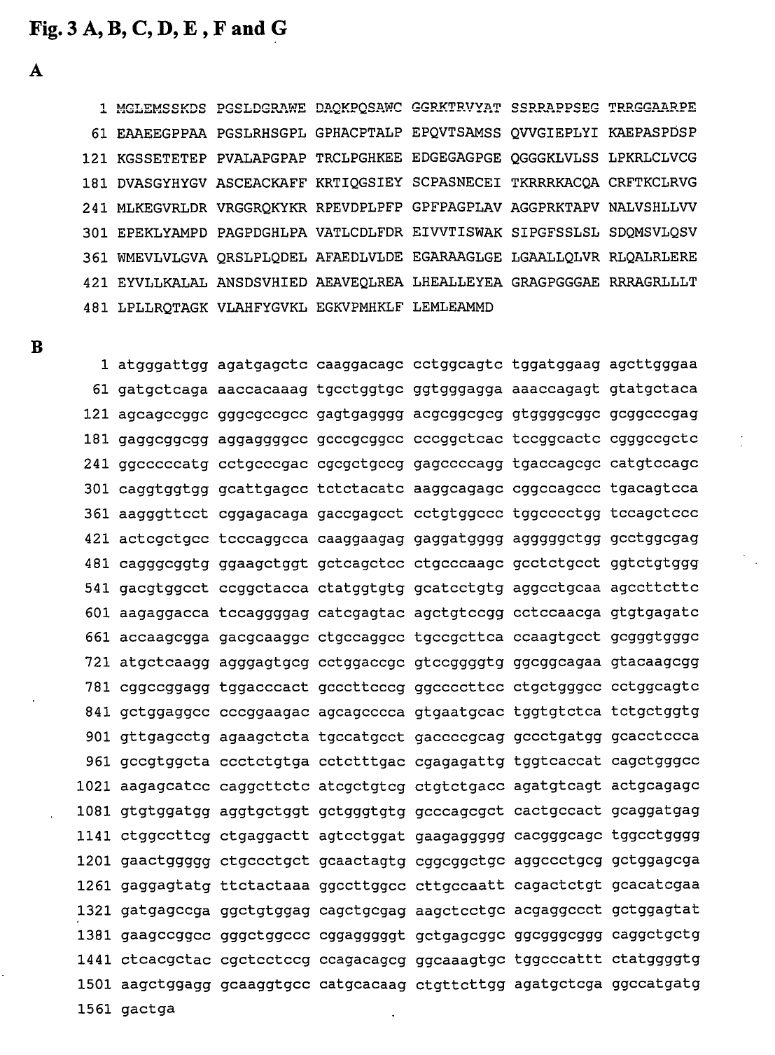

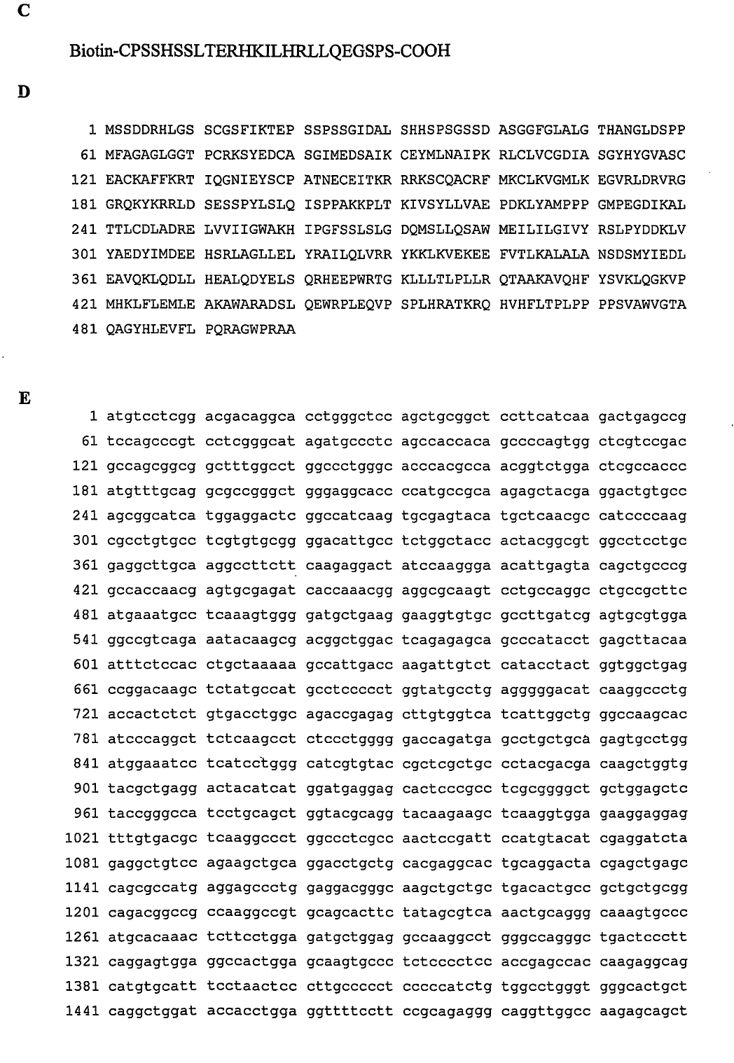

The present invention relates to a compound as described above wherein said compounds is capable of binding the NR3B1 receptor protein or a portion thereof according to SEQ ID NO. 1 (cf. FIG. 3 A to D) or a mammalian homologue thereof. The claimed compound can bind to the NR3B1 receptor protein or a portion thereof in a mixture comprising 10-200 ng of NR3B1 receptor protein or a portion thereof, preferably the ligand binding domain, 20 mM Tris/HCl at pH 7.9; 60 mM KCl; 5 mM MgCl₂; 160 ng/μl BSA in a total volume of preferably about 25 μl.

“A mammalian receptor protein homologue” of the protein according to SEQ ID NO. 1 as used herein is a protein that performs substantially the same task as NR3B1 does in humans and shares at least 40% sequence identity at the amino acid level, preferably 50% sequence identity at the amino acid level more preferably 65% sequence identity at the amino acid level, even more preferably 75% sequence identity at the amino acid level and most preferably over 85% sequence identity at the amino acid level.

The invention in particular relates to a method for prevention or treatment of a NR3B1 receptor protein or NR3B1 receptor protein homologue mediated disease or condition in a mammal comprising administration of a therapeutically effective amount of a compound according to the invention wherein the prevention or treatment is directly or indirectly accomplished through the binding of a compound according to the invention to the NR3B1 receptor protein or to the NR3B1 receptor protein homologue.

The term “mediated” herein means that the physiological pathway in which the NR3B1 receptor protein acts is either directly or indirectly involved in the disease or condition to be treated or prevented. In the case where it is indirectly involved it could be that, e.g. modulating the activity of NR3B1 by a compound according to the invention influences a parameter which has a beneficial effect on a disease or a condition. One such example is that modulation of NR3B1 activity leads to decreased levels of aromatase levels which in turn have a beneficial effect on the prevention and treatment of mammary cancer. Herein a condition is a physiological or phenotypic state which is desirably altered. One such example would be obesity which is not necessarily medically harmful but nonetheless a non desirable phenotypic condition. In a preferred embodiment of the invention the method for prevention or treatment of a NR3B1 receptor protein mediated disease or condition is applied to a human. This may be male or female.

Pharmaceutical compositions generally are administered in an amount effective for treatment or prophylaxis of a specific condition or conditions. Initial dosing in human is accompanied by clinical monitoring of symptoms, such symptoms for the selected condition. In general, the compositions are administered in an amount of active agent of at least about 100 μg/kg body weight. In most cases they will be administered in one or more doses in an amount not in excess of about 20 mg/kg body weight per day. Preferably, in most cases, doses is from about 100 μg/kg to about 5 mg/kg body weight, daily.

For administration particularly to mammals, and particularly humans, it is expected that the daily dosage level of active agent will be 0.1 mg/kg to 10 mg/kg and typically around 1 mg/kg.

By “therapeutically effective amount” is meant a symptom-alleviating or symptom-reducing amount, a cholesterol-reducing amount, a fatty acid absorption blocking amount, a protein and/or carbohydrate digestion-blocking amount, a MCAD modulating amount or a thyroid receptor modulating amount or a osteopontin modulating amount and/or a aromatase modulating amount of a compound according to the invention.

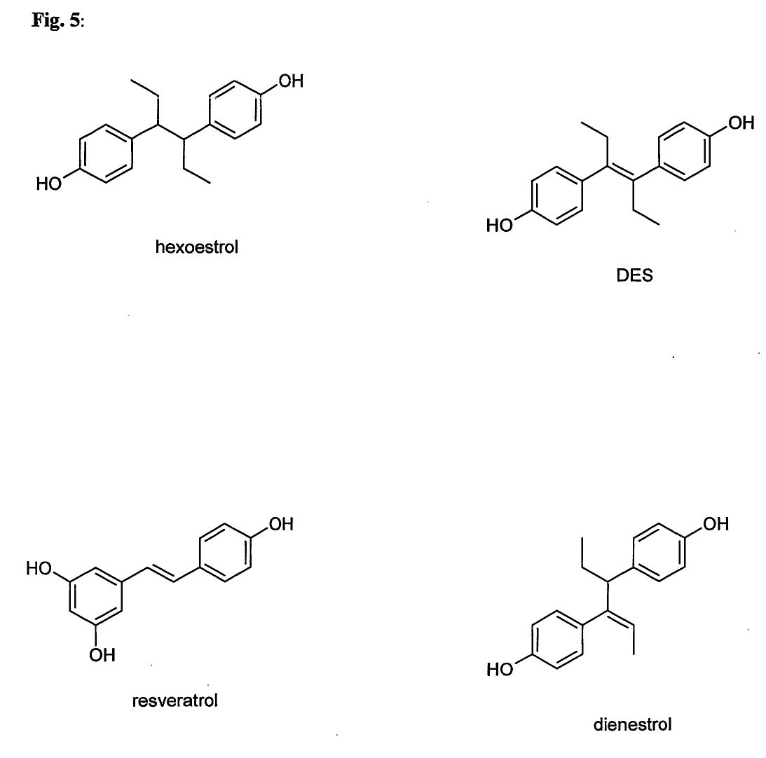

The Estrogen receptor related receptor alpha is a nuclear receptor which modulates genes upon binding to the promoter region of target genes in a homodimeric or monomeric fashion or as heterodimers with the ERa. The relevant physiological ligands of NR3B1 are unknown. The present compounds according to the invention have been demonstrated to have a high binding efficacy [binding coefficients measured as IC50 in the range 1 to 10 μM] as well as antagonistic and/or agonistic properties. Consequently they may be applied to regulate genes that participate in estrogen regulated gene networks as well as other downstream regulated genes. Examples of such genes are Lactoferrin, MCAD, Aromatase, PS2 and SHP and the function of such genes is associated but not limited to cancer, osteogenesis, obesity, energy metabolism, lipid absorption, cholesterol biosynthesis, cholesterol transport or binding, bile acid transport or binding, proteolysis, amino acid metabolism, glucose biosynthesis, protein translation, electron transport, and hepatic fatty acid metabolism. The ERR's often function in vivo as homodimers or as monomers. Published ERR antagonists such as the DES (See FIG. 5) are known to influence the regulation of various genes. Genes found to be regulated by DES can be found in FIG. 6. Thus, the invention also concerns a method of modulating a gene whose expression is regulated by the NR3B1 receptor in a mammal comprising administration of a therapeutically effective amount of a compound according to the invention to said mammal.

It is known that the orphan receptor ERRa can bind the response element of the aromatase gene (Yang C 2002 Oncogene 21, 2854-2863). Other genes controlled via ERRa comprise the medium chain acetyl dehydrogenase MCAD, Thyroid receptor alpha, osteopontin, PS2 and lactoferrin.

The compounds according to the invention may be used as medicaments, in particular for the manufacture of a medicament for the prevention or treatment of a NR3B1 receptor protein or NR3B1 receptor protein homologue mediated disease or condition in a mammal wherein the prevention or treatment is directly or indirectly accomplished through the binding of the compound according to the invention to the NR3B1 receptor protein or NR3B1 receptor protein homologue. These pharmaceutical compositions contain 0.1% to 99.5% of the compound according to the invention, more particularly 0.5% to 90% of the compound according to the invention in combination with a pharmaceutically acceptable carrier.

The invention relates also to the use of a compound according to the invention for the manufacture of a medicament for the prevention or treatment of a NR3B1 receptor protein mediated disease or condition wherein the mammal described above is a human. The medicament may be used for regulating the growth of cancer cells in a mammal preferentially a human by modulating the NR3B1 receptor, for regulating cancer, osteoporosis, lipid disorders or a cardiovascular disorder in humans or influencing fertility and reproductive health.

The further concerns the use of a compound according to the invention for the manufacture of a medicament capable for blocking in a mammal, preferentially a human the fatty acid absorption in the intestine. Further the claimed compound may be used for the manufacture of a medicament for treating obesity in humans and for modulating a gene whose expression is regulated by the NR3B1 receptor (see details above and figures). The invention further concerns the use of a compound according to the invention for the manufacture of antitumor medicaments. The antitumor effects of such medicaments could be excerted by selective inhibition of cell proliferation and induction of apotptosis of tumour cells.

The invention shall now be further described in the following examples, without being limited thereto. In the accompanying figure and the sequence protocol,

SEQ ID NO. 1: shows the peptide sequence of ERRa,

SEQ ID NO. 2: shows the nucleotide sequence of the region that encodes for ERRa,

SEQ ID NO. 3: depicts the sequence of the biotinylated SRC1 peptide,

SEQ ID NO. 4: shows primer ERRE_{—}1, used in Examples 3 and 4,

SEQ ID NO. 5: shows primer ERRE_{—}2, used in Examples 3 and 4,

SEQ ID NO 6 is the protein sequence of the ERRb protein,

SEQ ID NO 7 is the mRNA sequence coding for the ERRb protein,

SEQ ID NO 7 is the protein sequence of the ERRg protein, and

SEQ ID NO 8 is the mRNA sequence of the ERRg protein.

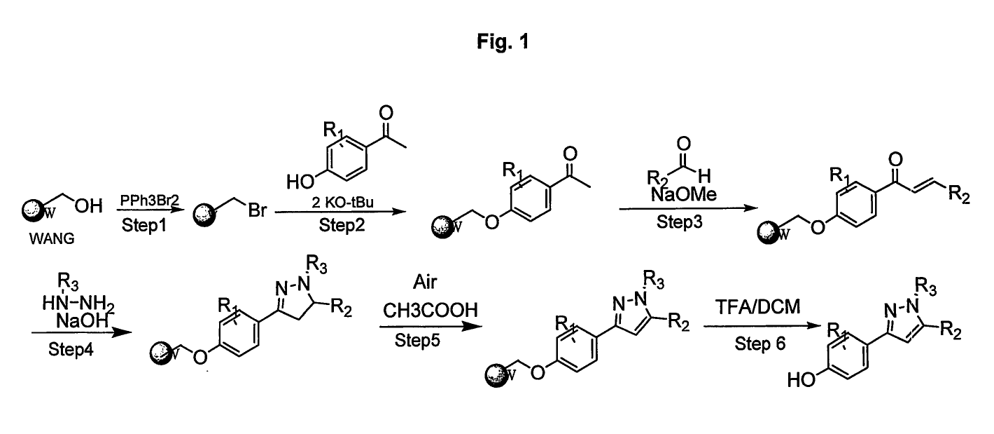

FIG. 1 shows the synthesis of the compounds according to the invention as also described in Example 2.

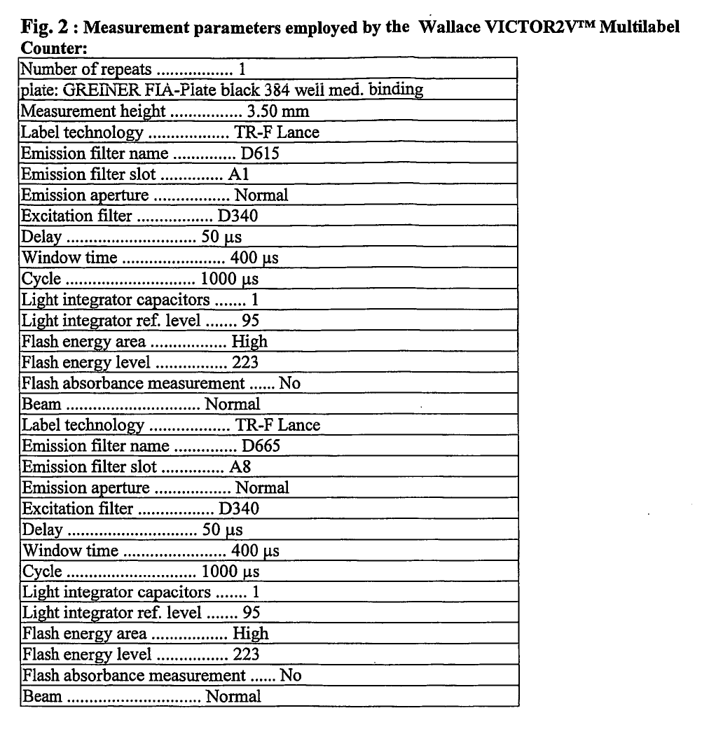

FIG. 2 shows the measurement parameters employed by the Wallace VICTOR2V™ Multilabel Counter which was used for measuring the EC₅₀ values

FIG. 3 A shows SEQ ID NO. 1 which is the protein sequence of the ERRa protein a portion of which was used for cloning as described in the examples. FIG. 3 B shows SEQ ID NO. 2 which is the mRNA sequence of the ERRa protein. FIG. 3 C shows SEQ ID NO. 3 which is the sequence of the biotinylated SRC1 peptide. FIG. 3D shows SEQ ID NO 4 which is the protein sequence of the ERRb protein a portion of which was used according to the example described for ERRa. FIG. 3E shows SEQ ID NO 5 which is the mRNA sequence coding for the ERRb protein. FIG. 3F show SEQ ID NO 6 which is the protein sequence of the ERRg protein, a portion of which was used for cloning according to the example described for ERRa. FIG. 3G shows SEQ ID NO 7 which is the mRNA sequence of the ERRg protein.

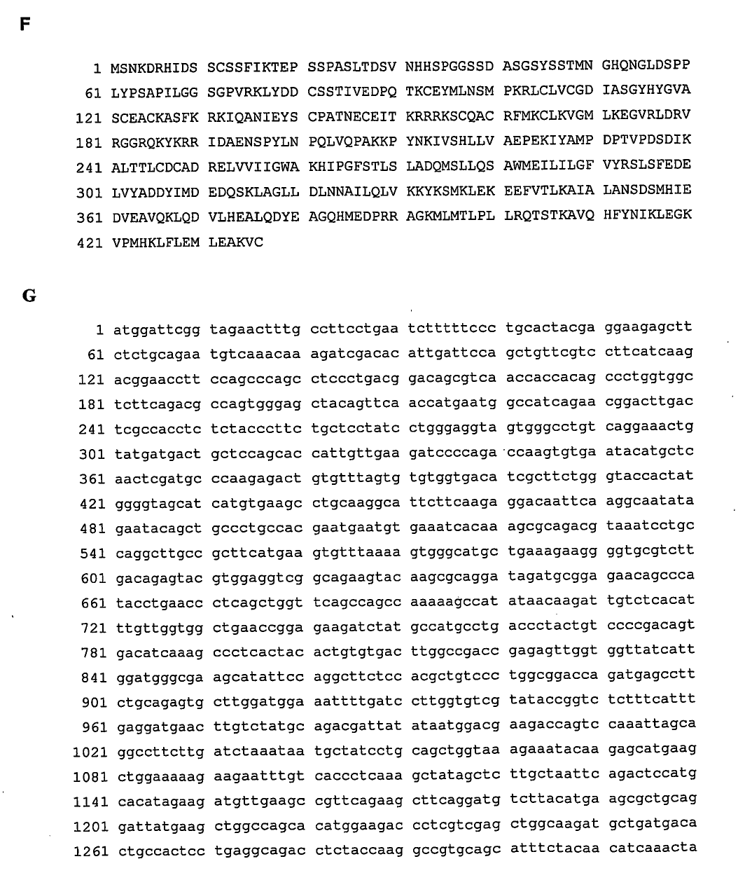

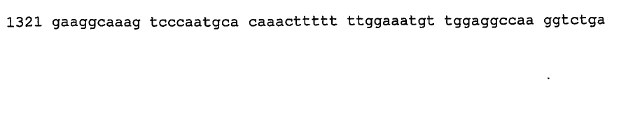

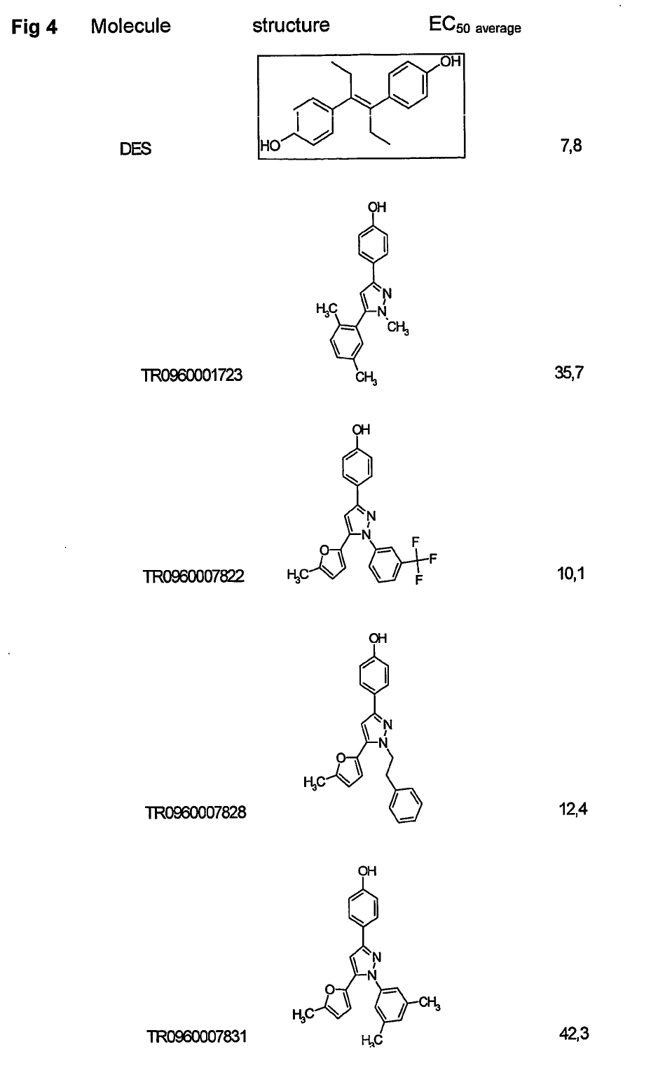

FIG. 4 shows the internal molecular name used by the applicant (MOLNAME) as well as the corresponding structures of preferred compounds according to the invention. The figure further shows their respective EC₅₀ values (EC50 AVG) as established according to the example 1 in multiple experiments (see above), as well as their respective estimated average efficacy (% activity relative to DES control antagonist).

FIG. 5 shows various known ERRa ligands. It is apparent from their structures that the inventors have identified novel compounds which are structurally not related to these known ligands.

FIG. 6 shows various genes that have been found to be regulated through binding of an ERRa protein.

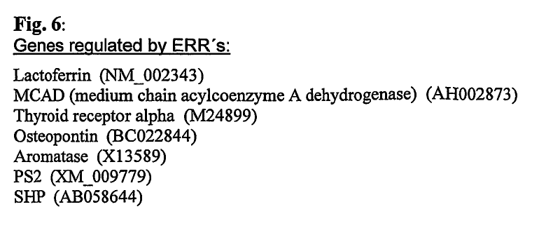

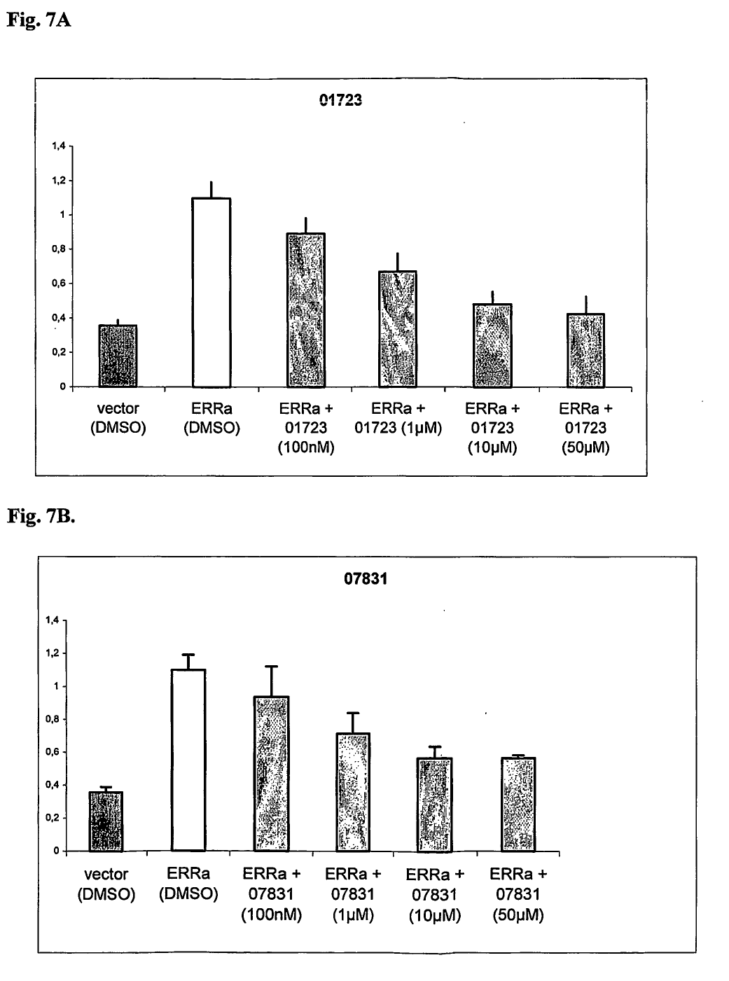

FIG. 7 shows a dose-dependent transrepression (EC50˜5 μM) of the ERRa reporter gene by ERRa by 01723 (A) or 07831 (B).

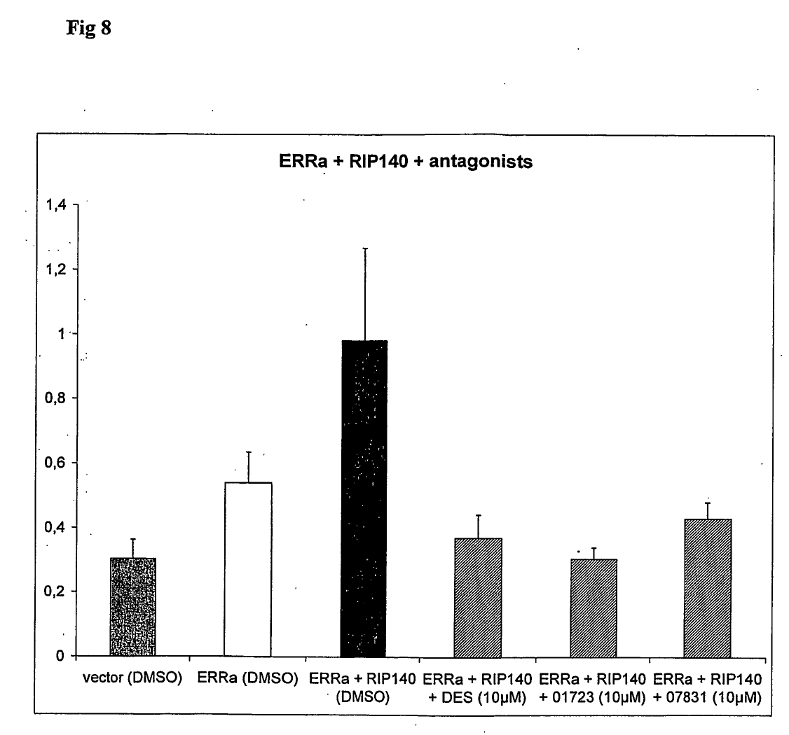

FIG. 8 shows the stimulation of the ERRa driven reporter gene activity by RIP140 and repression of this RIP140 dependent activity by 10 μM 01723, 07831 or DES.

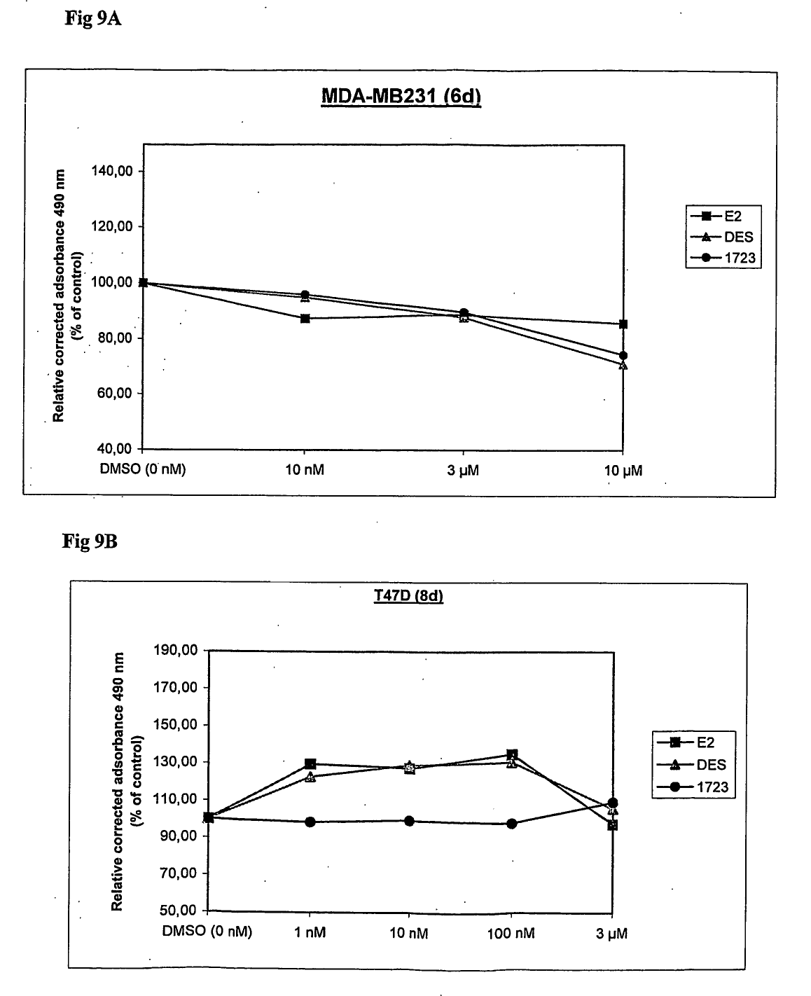

FIG. 9 shows the ERRa ligand DES and the compound 01723, inhibit growth of the ER negative breast cancer cell line MDA-MB-231 (FIG. 9A). The compound 01723 does not induce proliferation of the ER positive breast cancer cell line T47D, while both E2 (estradiol) and DES stimulate the growth of T47D cells at indicated concentrations (FIG. 9B).

### EXAMPLES

### Example 1

In Vitro Screening for Compounds Which Influence ERRa Binding to Coactivators

For screening purposes a fragment of the open reading frame of human ERR alpha (NR3B1-(Acc. No: NM_{—}004451)) encoding aminoacids 187-472 was amplified by standard RT PCR procedures (see figures; SEQ ID NO. 1 and 2). Starting material was total RNA derived from human uterus. The resulting cDNA obtained after reverse transcription was subsequently cloned using the Gateway™ recombination technology (Invitrogen, USA) into the expression plasmid pDest15 (Invitrogen, USA). This construct was used to express a recombinant GST-ERRa fusion protein in E. coli (BL21 strain). For E. coli expression of both constructs, plasmid DNA was transformed into chemically competent E. coli BL21 (Invitrogen, USA) and cells were grown to an OD600 of 0.4-0.7 before expression was induced by addition of 0.5 mM IPTG according instructions of the manufacturer (Invitrogen). After induction for 8 hours at 30° C. cells were harvested by centrifugation for 10 minutes at 5000×g. Fusion proteins were affinity purified using Glutathion sepharose (Pharmacia) or Ni-NTA Agarose (QIAGEN) according to the instructions of the respective manufacturer. Recombinant proteins were dialyzed against 20 mM Tris/HCL pH 7.9; 60 mM KCl; 5 mM MgCl₂; 1 mM DTT, 0.2 mM PMSF; 10% glycerol.

For screening of compound libraries as provided for by the methods shown below in the examples for substances which influence the ERRa/SRC1 interaction, the Perkin Elmer LANCE technology was applied. This method relies on the binding dependent energy transfer from a donor to an acceptor fluorophore attached to the binding partners of interest. For ease of handling and reduction of background from compound fluorescence LANCE technology makes use of generic fluorophore labels and time resoved detection (for detailed description see Hemmilä I, Blomberg K and Hurskainen P, Time-resolved resonance energy transfer (TRFRET) principle in LANCE, Abstract of Papers Presented at the 3 rd Annual Conference of the Society for Biomolecular Screening, September, California (1997)).

For screening, 80 ng of biotinylated SRC1 peptide and 10-200 ng of GST-ERRa fragment were combined with 0.5-2 nM LANCE Eu-(W1024) labelled anti-GST antibody (Perkin Elmer) and 0.5-2 μg of Highly fluorescent APC-labelled streptavidin (Perkin Elmer) in the presence of 50 μM of individual compounds to be screened in a total volume of 25 μl of 10 mM Hepes pH 7.9; 550 mM NaCl; 2 mM MgCl₂; 40 ng/μl BSA. DMSO content of the samples was kept below 1%. Samples were incubated for a minimum of 60 minutes in the dark at room temperature in FIA-Plates black 384well med. binding (Greiner).

The LANCE signal was detected by a Perkin Elmer VICTOR2V™ Multilabel Counter applying the detection parameters listed in FIG. 2. The results were visualized by plotting the ratio between the emitted light at 665 nm and at 615 nm. For every batch of recombinant proteins amount of proteins and labeling reagents giving the most sensitive detection of hits was determined individually by analysis of dose response curves for DES.

### Example 2

Experimental Procedure for the Preparation of the Compounds According to the Invention (see also FIG. 1)

Step 1. General Procedure for Preparation of Br-Wang Resin

1.6 g of Wang resin (1.28 mmol/g, 2.0 mmol/bag) was placed in a porous polypropylene packets (Tea-bag, 60 mm×50 mm, 65μ), sealed and transferred to a 125 ml PP bottle. A freshly prepared solution of PPh₃Br₂ (6.1 mmol, 3.0 equivalents, 0.15 M) in DCM (40 mL) was added to each packet. After shaking for 4-6 hours at room temperature, the packet was washed with DCM (5×80 ml) and diethyl ether (4×80 ml). The packet was dried overnight under vacuum to afford off-white resin.

Step 2. Reaction of Acetophenones with Br-Wang Resin.

Each packet containing freshly prepared Br-Wang resin was transferred to an appropriate glass bottle, to which an Acetophenone (20 mmol, 10 equivalents, 0.2 M), anhydrous DMA (100 ml) and KOtBu (20 mmol, 10 equivalents, 0.2 M) were added sequentially. After heating at 50° C. for 24 hours, the packet was washed alternatively with DMF (3×80 ml) and MeOH (2×80 ml) followed by DCM (2×80 ml) and MeOH (3×80 ml). The packet was air-dried overnight to afford off-white to pale brown resin, depending on the Acetophenone used in the synthesis.

Step 3. Reaction of Aldehydes with Wang Resin-Bound Acetophenones.

Each packet of Acetophenone-Wang resin was transferred to a 250 mL PP bottle, to which a solution of NaOMe (40 mmol, 20 eq, 0.25 M) in 50% THF-MeOH (160 ml) and an Aldehyde (40 mmol, 20 equivalents, 0.25 M) were added sequentially. After shaking at room temperature for 3 days, the packet was washed several times with MeOH (3×80 ml) and alternatively with DMF (80 ml) and MeOH (80 ml) for 3 cycles, followed by washes of DCM (2×80 ml) and MeOH (3×80 ml). The packet was air-dried overnight to afford a resin-bound chalcone, that varied in color from yellow to dark red depending on the aldehyde used.

Step 4: Reaction of Hydrazines with Wang Resin-Bound Chalcone.

The tea bag containing the Chalcone on Wang resin from step was cut open and the resin was equally distributed into 40 wells of a microtiter plate (approx. 50 mg, 0.0575 mmol for each well). The appropriate Hydrazine (0.575 mmol, 10 eq, 0.32 M), NaOH (1.15 mmol, 20 eq, 0.65 M) and MeOEtOH (1.8 mL) were mixed for 2 hrs at room temperature. The supernatant (hydrazine solution) was then added to the corresponding well. The plate was tightly capped, gently shaken and incubated at 75° C. for 24 hours. Each plate was washed alternatively with DMSO (5×1 mL/well), DMF (6×1 mL/well), and MeOH (8×1 mL/well). The plate was air-dried overnight and under vacuum for 4 hours.

Step 5. Air Oxidation of Pyrazoline in Acetic Acid:

To each well of microtiter plate containing the pyrazoline resins was added 1 ml of acetic acid which was bubbled with air for 20 minutes. The plates were tightly capped, gently shaken, and incubated at 75° C. for 48 hours. Each plate was washed alternatively with DMF (6×1 mL/well) and MeOH (8×1 mL/well). The plate was air-dried overnight. and under vacuum for 4 hours.

Step 6. Cleavage from Linker and Extraction

To dry microtiter plates containing the pyrazole-resins was added 0.5 mL of 20% TFA/DCM to each well. The plates were capped and placed on a shaker at room temperature for 3 h. The plates were transferred to a GENEVAC to remove the volatile TFA/DCM solution. The plates were extracted with AcOH and the extract frozen and lyophilised to afford the title compounds. All of the final products were analysed by HPLC/MS using ELSD detection to determine purity.

### Example 3

This example illustrates that a compound according to the invention (experiments shown were done with MOLSTRUCTURE 01723 and 07831 (see FIG. 4 for structural formula) can mediate repression of ERRa mediated transcription in HEK293 cells.

HEK293-were transiently transfected with the pTRexDest30 (Invitrogen) derivatives pTRexDest30-hERRa and the pGL2promoter (Promega) derivative pGL2promoter-ERR-RE2. The Renilla-Luciferase pRL-CMV Vector (Promega) was included as a control for transfection efficiency.

The full length human ERRa (accession NM_{—}004451) was cloned into the pTRexDest30 applying the manufacturer protocols for the Gateway™ system (Invitrogen). The pGL2promoter-ERR-RE2 (ERRa reporter gene construct) was generated by annealing oligonucleotides ERRE_{—}1 (5′-TCGAGGCGATTTGTCAAGGTCACACAGTA-3′) (SEQ ID No. 4) and ERRE_{—}2 (5′-TCGATACTGTGTGACCTTGACAAATCGCC-3′) (SEQ ID No. 5) and cloning them into the Xho I site of the pGL2promoter (Promega). The oligonucleotides contain a consensus ERR response element (underlined). Sequencing confirmed the presence of two copies of the ERR RE.

Luciferase reporter activity was measured in triplicates from extracts of cells after incubating cells in culture medium (phenolred-free DMEM [Gibco-BRL]+10% charcoal treated FBS [Perbio Science GmbH]) for 16 hours (5% CO₂, 37° C.) containing 0.001% DMSO (control) or 0.001% DMSO with increasing concentrations of 01723 or 07831.

A dose-dependent repression (EC50˜1-5 μM) of the reporter gene driven by ERRa was observed for the compounds TR0960001723 (FIG. 7A) and TR0960007831 (FIG. 7 B). Variations of tripicate measurements are indicated.

### Example 4

This example illustrates that a compound according to the invention (experiments shown were done with MOLSTRUCTURE TR0960001723 and TR0960007831 and DES as a control (see FIGS. 4 and 5 for structural formula) can mediate repression of ERRa mediated transcription in HEK293 cells.

HEK293-were transiently transfected with the pTRexDest30 (Invitrogen) derivatives pTRexDest30-hERRa and pTRexDest30-hRIP140 and the pGL2promoter (Promega) derivative pGL2promoter-ERR-RE2. The Renilla-Luciferase pRL-CMV Vector (Promega) was included as a control for transfection efficiency.

The full length human ERRa (accession NM_{—}004451) and RIP 140 (X84373) were cloned into the pTRexDest30 applying the manufacturer protocols for the Gateway™ system (Invitrogen).

The pGL2promoter-ERR-RE2 was generated by annealing oligonucleotides ERRE_{—}1 (5′-TCGAGGCGATTTGTCAAGGTCACACAGTA-3′) (SEQ ID No. 4) and ERRE_{—}2 (5′-TCGATACTGTGTGACCTTGACAAATCGCC-3′) (SEQ ID No. 5) and cloning them into the Xho I site of the pGL2promoter (Promega). The oligonucleotides contain a consensus ERR response element (underlined). Sequencing confirmed the presence of two copies of the ERR RE.

Luciferase reporter activity was measured in triplicates from extracts of cells after incubating cells in culture medium (phenolred-free DMEM [Gibco-BRL]+10% charcoal treated FBS [Perbio Science GmbH]) for 16 hours (5% CO₂, 37° C.) containing 0.001% DMSO (control) or 0.001% DMSO with increasing concentrations of 01723 or 07831.

The activity of the ERRa reporter gene construct in HEK293 cells is enhanced when ERRa is cotransfected. This activity is further enhanced, when RIP140 is cotransfected in addition. The observed enhancement of ERRa activity is significantly reduced, when 10 μM of either DES or TR0960001723 or TR0960007831 are added to the medium (see FIG. 8)

### Example 5

This example illustrates that a compound according to the patent (MOLSTRUCTURE TR0960001723; see FIG. 4 for structural formula) can inhibit proliferation of an estrogen receptor negative cell line (MDA-MB-231).

Cell proliferation assays were performed with MDA-MB-231 cells (ER negative) and T47D cells (ER positive) in the presence of 17β-estradiol (E2), DES, TR0960001723 or TR0960007831. Cells were seeded in 96-well plates at a density of 15,000 (MDA-MB-231) or 10,000 (T47D) cells/100 μl/well in phenol red-free DMEM (Gibco-BRL) containing 10% (MDA-MB-231) or 2% (T47D) charcoal dextran treated FBS (Perbio Science GmbH). Treatment media containing either 0.1% DMSO (control) or 0.1% DMSO with increasing concentrations of compounds (as indicated) were added on the following day and replaced at 72 h intervals until the end of the experiment. At 6 days (MDA-MB-231) or 8 days (T47D) after initiation of the treatment a colorimetric proliferation assay was performed using CellTiter 96 Aqueous nonradioactive proliferation assay as directed by the manufacturer (Promega). Cell proliferation rates were measured as absorbance of the formazan product at 490 nm. Values are expressed as percentage of control (DMSO) and represent the means of three replicates.

As observed, DES and 17β-estradiol stimulated T47D cell proliferation at 1 nM to 100 nM, but TR0960001723 had no effect (FIG. 9B). In contrast, DES and TR0960001723 inhibited proliferation of MDA-MB-231 cells whereas 17β-estradiol did not (FIG. 9A).

1. A compound according to formula (1), or pharmaceutical acceptable salts or solvates thereof, 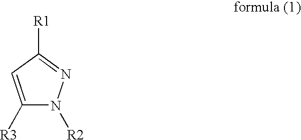
wherein:
R₁ is phenyl, substituted phenyl, C₅ to C₆ heteroaryl, C₅ to C₆ substituted heteroaryl, napthyl or substituted napthyl,
R₂ is H, C₁ to C₈ alkyl, C₁ to C₇ acyl, C₁ to C₇ substituted acyl, C₁ to C₈ substituted alkyl, C₇ to C₁₂ alkylphenyl, C₇ to C₁₂ substituted phenylalkyl, C₃ to C₈ cycloalkyl, C₃ to C₈ substituted cycloalkyl, C₅ to C₆ heteroaryl, or [C₅ to C₆]-heteroaryl-(C₁ to C₆)-alkyl, and
R₃ is H, C₁ to C₈ alkyl, C₁ to C₈ substituted alkyl, C₇ to C₁₂ alkylphenyl, C₇ to C₁₂ substituted phenylalkyl, halogen, C₁ to C₈ alkoxy, furanyl, substituted furanyl, thiazyl, substituted thiazyl, carboxy, ester, amide or C₁ to C₈ aminoacyl.

2. The compound according to claim 1, or pharmaceutical acceptable salts or solvates thereof, wherein:
R₁ is phenyl, substituted phenyl, C₅ to C₆ heteroaryl, or C₅ to C₆ substituted heteroaryl,
R₂ is H, CH₃, substituted alkyl or substituted phenyl, and
R₃ is substituted phenyl, C₅ to C₆ heteroaryl or C₅ to C₆ substituted heteroaryl.

3. The compound according to claim 1, or pharmaceutical acceptable salts or solvates thereof, wherein:
R₁ is substituted phenyl,
R₂ is CH₃ or substituted alkyl, and
R₃ is substituted phenyl or substituted C₅ heteroaryl.

4. The compound according to claim 1, having the following formula (2) 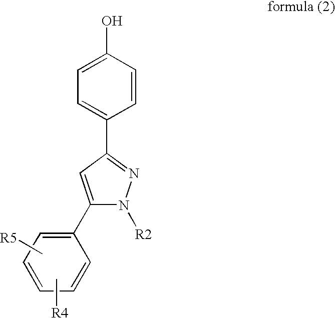
wherein:
R₂ is H, C₁ to C₈ alkyl, C₁ to C₈ substituted alkyl, C₇ to C₁₂ alkylphenyl or C₇ to C₁₂ substituted phenylalkyl,
R₄ is H, C₁ to C₈ alkyl, halogen, C₁ to C₈ alkoxy, carboxy, ester, amide or C₁ to C₈ aminoacyl, and
R₅ is H, C₁ to C₈ alkyl, halogen, C₁ to C₈ alkoxy, carboxy, ester, amide or C₁ to C₈ aminoacyl.

5. The compound according to claim 1 having the following formula (3) 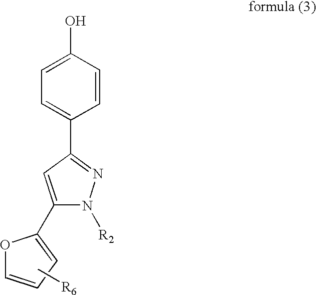
wherein:
R₂ is H, C₁ to C₇ acyl, C₁ to C₇ substituted acyl, phenyl, substituted phenyl, C₅ to C₆ heteroaryl, C₅ to C₆ substituted heteroaryl, napthyl or substituted napthyl,
R₆ is H, C₁ to C₈ alkyl, C₁ to C₈ substituted alkyl, C₇ to C₁₂ alkylphenyl, C₇ to C₁₂ substituted phenylalkyl, carboxy, ester, amide C₁ to C₈ aminoacyl, or C₁ to C₈ alkoxy.

6. The compound according to claim 1 having the following formula (4) 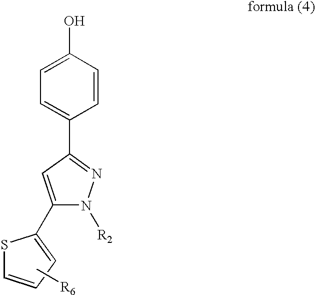
wherein:
R₂ is H, C₁ to C₇ acyl, C₁ to C₇ substituted acyl, phenyl, substituted phenyl, C₅ to C₆ heteroaryl, C₅ to C₆ substituted heteroaryl, napthyl or substituted napthyl,
R₆ is H, C₁ to C₈ alkyl, C₁ to C₈ substituted alkyl, C₇ to C₁₂ alkylphenyl, C₇ to C₁₂ substituted phenylalkyl, carboxy, ester, amide, C₁ to C₈ aminoacyl, or C₁ to C₈ alkoxy.

7. The compound according to claim 1 wherein said compound is capable of binding the NR3B1 receptor protein or a portion thereof according to SEQ ID NO. 3 or a mammalian homologue thereof.

8. The compound according to claim 1 wherein said compound is capable of modulating the activity of the NR3B1 receptor protein comprising antagonistic or agonistic effects.

9. A method for prevention or treatment of a NR3B1 receptor protein or NR3B1 receptor protein homologue mediated disease or condition in a mammal comprising administration of a therapeutically effective amount of a compound according to formula (1), or pharmaceutical acceptable salts or solvates thereof, 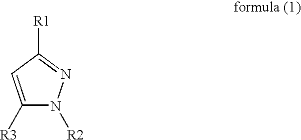
wherein:
R₁ is phenyl, substituted phenyl, C₅ to C₆ heteroaryl, C₅ to C₆ substituted heteroaryl, napthyl or substituted napthyl,
R₂ is H, C₁ to C₈ alkyl, C₁ to C₇ acyl, C₁ to C₇ substituted acyl, C₁ to C₈ substituted alkyl, C₇ to C₁₂ alkylphenyl, C₇ to C₁₂ substituted phenylalkyl, C₃ to C₈ cycloalkyl, C₃ to C₈ substituted cycloalkyl, C₅ to C₆ heteroaryl, or [C₅ to C₆]-heteroaryl-(C₁ to C₆)-alkyl, and
R₃ is H, C₁ to C₈ alkl, C₁ to C₈ substituted alkyl, C₇ to C₁₂ alkylphenyl, C₇ to C₁₂ substituted phenylalkyl, halogen, C₁ to C₈ alkoxy, furanyl, substituted furanyl, thiazyl, substituted thiazyl, carboxy, ester, amide or C₁ to C₈ aminoacyl
wherein the prevention or treatment is directly or indirectly accomplished through the binding of said compound to the NR3B1 receptor protein or to the NR3B1 receptor protein homologue.

10. The method for prevention or treatment of a NR3B1 receptor protein mediated disease or condition according to claim 9 wherein the mammal is a human.

11. A method for
i. regulating physiologies that are influenced by estrogenic response pathways in a mammal comprising modulating the activity of the NR3B1 receptor;
ii. treating in a mammal a disease which is directly or indirectly affected by estrogen levels,
iii. treating cancer, osteoporosis, obesity, lipid disorders or a cardiovascular disorder or influencing fertility and reproductive health in a mammal; and/or
iv. modulating the expression of a gene directly or indirectly controlled by NR3B1 in tissues of a mammal,
wherein said method comprises administering to a mammal in need of such regulation, treatment and/or modulation an effective amount of a compound according to formula (1), or pharmaceutical acceptable salts or solvates thereof, 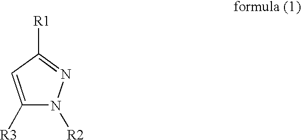
wherein:
R₁ is phenyl, substituted phenyl, C₅ to C₆ heteroaryl, C₅ to C₆ substituted heteroaryl, napthyl or substituted napthyl,
R₂ is H, C₁ to C₈ alkyl, C₁ to C₇ acyl, C₁ to C₇ substituted acyl, C₁ to C₈ substituted alkyl, C₇ to C₁₂ alkylphenyl, C₇ to C₁₂ substituted phenylalkyl, C₃ to C₈ cycloalkyl, C₃ to C₈ substituted cycloalkyl, C₅ to C₆ heteroaryl, or [C₅ to C₆]-heteroaryl-(C₁ to C₆)-alkyl, and
R₃ is H, C₁ to C₈ alkyl, C₁ to C₈ substituted alkyl, C₇ to C₁₂ alkylphenyl, C₇ to C₁₂ substituted phenylalkyl, halogen, C₁ to C₈ alkoxy, furanyl, substituted furanyl, thiazyl, substituted thiazyl, carboxy, ester, amide or C₁ to C₈ aminoacyl.

12-14. (canceled)

15. The method of claim 11, wherein said mammal is a human.

16. The method according to claim 15 for treating cancer, osteoporosis, lipid disorders or a cardiovascular disorder in humans or influencing fertility and reproductive health.

17. The method according to claim 11 wherein the expression of genes comprising aromatase, MCAD, thyroid receptor alpha, osteopontin, PS2, lactoferrin is modulated.

18-26. (canceled)

27. A pharmaceutical composition comprising a compound according to formula (1), or pharmaceutical acceptable salts or solvates thereof, 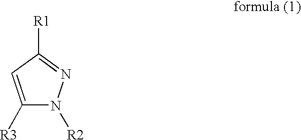
wherein:
R₁ is phenyl, substituted phenyl, C₅ to C₆ heteroaryl, C₅ to C₆ substituted heteroaryl, napthyl or substituted napthyl,
R₂ is H, C₁ to C₈ alkyl, C₁ to C₇ acyl, C₁ to C₇ substituted acyl, C₁ to C₈ substituted alkyl, C₇ to C₁₂ alkylphenyl, C₇ to C₁₂ substituted phenylalkyl, C₃ to C₈ cycloalkyl, C₃ to C₈ substituted cycloalkyl, C₅ to C₆ heteroaryl, or [C₅ to C₆]-heteroaryl-(C₁ to C₆)-alkyl, and
R₃ is H, C₁ to C₈ alkyl, C₁ to C₈ substituted alkyl, C₇ to C₁₂ alkylphenyl, C₇ to C₁₂ substituted phenylalkyl, halogen, C₁ to C₈ alkoxy, furanyl, substituted furanyl, thiazyl, substituted thiazyl, carboxy, ester, amide or C₁ to C₈ aminoacyl;
together with a pharmaceutical carrier.
